(19) United States
(12) Patent Application Publication
(10) Pub. No.: US 2014/0155983 A1
(43) Pub. Date: Jun. 5, 2014

(54) METHOD AND APPARATUS FOR EFFECTING AN AORTIC VALVE BYPASS, INCLUDING THE PROVISION AND USE OF A T-STENT FOR EFFECTING A DISTAL ANASTOMOSIS FOR THE SAME

(71) Applicant: Correx, Inc., Waltham, MA (US)

(72) Inventors: Richard M. Beane, Hingham, MA (US); John W. Brown, Indianapolis, IN (US); James Alan Crunkleton, Weston, MA (US); James S. Gammie, Stevenson, MD (US); Anthony G. Liepert, Lincoln, MA (US); Joseph L. Smith, Jr., Concord, MA (US)

(21) Appl. No.: 13/915,875

(22) Filed: Jun. 12, 2013

Related U.S. Application Data
(63) Continuation of application No. 12/829,181, filed on Jul. 1, 2010, now abandoned.
(60) Provisional application No. 61/222,183, filed on Jul. 1, 2009.

(51) Int. Cl. A61F 2/24; A61F 2/856; A61F 2/07; A61B 17/11

(52) U.S. Cl. CPC A61F 2/2418; A61B 17/11; A61F 2/856; A61F 2/07
USPC 623/1.13; 606/153; 623/1.35; 623/2.17

(57) ABSTRACT

A connector for joining first and second hollow structures, comprising a fluid-constraining tube having a fluid-constraining neck extending therefrom, wherein the tube and the neck each comprise a lumen having first and second openings, the neck being joined to the tube such that fluid entering the first opening of the tube can exit the second opening of the tube, and fluid entering the first opening of the neck can exit the second opening of the tube; at least the portions of the tube adjacent to the first and second openings of the tube being biased radially outwardly so that they normally assume a radially-expanded configuration, but being capable of being restrained in a radially-contracted configuration, wherein the tube is sized so that, when it is in its radially-expanded configuration, it has an outer diameter which is larger than the inner diameter of the second hollow structure.

### REFERENCE TO PENDING PRIOR PATENT APPLICATION

This patent application claims benefit of pending prior U.S. Provisional Patent Application Ser. No. 61/222,183, filed Jul. 1, 2009 by Richard M. Beane et al. for DISTAL ANASTOMOSIS USING A T STENT (Attorney's Docket No. CORREX-46 PROV), which patent application is hereby incorporated herein by reference.

### FIELD OF THE INVENTION

This invention relates to surgical methods and apparatus in general, and more particularly to surgical methods and apparatus for effecting an aortic valve bypass.

### BACKGROUND OF THE INVENTION

Aortic valve bypass is a proven procedure for relieving critical aortic stenosis. This procedure requires the installation of a bypass conduit, having a prosthetic valve therein, between the interior of the left ventricle and the descending aorta. This approach allows blood to be pumped between the left ventricle and the descending aorta without requiring removal of the dysfunctional native aortic valve. See FIG. 1.

In an aortic valve bypass procedure, the connection of the bypass conduit to the descending aorta is commonly referred to as the “distal anastomosis”, and it is currently one of the more difficult and time-consuming elements of an aortic valve bypass procedure.

Currently, in order to effect the distal anastomosis, it is necessary to perform an anterior lateral thoracotomy of approximately six inch length in order to gain access to the descending aorta. The descending aorta is side-clamped so as to engage, but not occlude, the artery. Then a longitudinal slit is made in the clamped portion of the artery wall, and a graft (e.g., the distal end of the bypass conduit, or an element which is to be secured to the distal end of the bypass conduit), typically 14-20 mm in diameter, is sutured in place substantially perpendicular to the side wall of the descending aorta so as to establish the desired fluid connection. Once the perimeter of the graft has been secured to the slit aortic wall, the side clamp can be released and the distal anastomosis is complete.

Aortic valve bypass is not currently a common procedure, at least in part due to the relatively difficult and time-consuming nature of the distal anastomosis. Furthermore, aortic valve bypass cannot currently be considered to be a minimally invasive procedure, due to the need to provide an anterior lateral thoracotomy of approximately 6 inch length. However, reducing the size of the thoracotomy with the current procedure is problematic at best, since reduced access to the descending aorta makes cross-clamping and suturing all the more difficult and time-consuming. Also, when the ribs are spread to create access to the thoracic cavity, the ribs can sometimes fracture, thereby causing additional trauma to the patient.

Consequently, there is a need for an improved method and apparatus for effecting the distal anastomosis in an aortic valve bypass procedure.

### SUMMARY OF THE INVENTION

These and other objects of the present invention are addressed by the provision and use of a novel method and apparatus for effecting the distal anastomosis in an aortic valve bypass procedure.

In one form of the invention, there is provided a connector for joining a first hollow structure to the side wall of a second hollow structure, the connector comprising:

a fluid-constraining tube having a fluid-constraining neck extending therefrom, wherein the tube comprises a lumen having a first opening and a second opening and the neck comprises a lumen having a first opening and a second opening, the neck being joined to the tube so that the neck is in fluid communication with the tube intermediate the length of the tube, such that fluid entering the first opening of the tube can exit the second opening of the tube, and fluid entering the first opening of the neck can exit the second opening of the tube;

at least the portions of the tube adjacent to the first opening of the tube and the second opening of the tube being biased radially outwardly so that they normally assume a radially-expanded configuration, but being capable of being restrained in a radially-contracted configuration, wherein the tube is sized so that, when it is in its radially-expanded configuration, it has an outer diameter which is larger than the inner diameter of the second hollow structure.

In another form of the invention, there is provided a method for joining a first hollow structure to the side wall of a second hollow structure, the method comprising:

providing a connector comprising:
a fluid-constraining tube having a fluid-constraining neck extending therefrom, wherein the tube comprises a lumen having a first opening and a second opening and the neck comprises a lumen having a first opening and a second opening, the neck being joined to the tube so that the neck is in fluid communication with the tube intermediate the length of the tube, such that fluid entering the first opening of the tube can exit the second opening of the tube, and fluid entering the first opening of the neck can exit the second opening of the tube;
at least the portions of the tube adjacent to the first opening of the tube and second opening of the tube being biased radially outwardly so that they normally assume a radially-expanded configuration, but being capable of being restrained in a radially-contracted configuration, wherein the tube is sized so that, when it is in its radially-expanded configuration, it has an outer diameter which is larger than the inner diameter of the second hollow structure;
restraining the tube in its radially-contracted configuration;
selecting a location on the side wall of the second hollow structure;
forming an opening in the side wall of the second hollow structure at the selected location;
positioning the connector so that the tube resides within the interior of the second hollow structure and the neck extends out of the side wall of the second hollow structure;
allowing the tube to expand back into its radially-expanded configuration; and
connecting the neck to the first hollow structure.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing an aortic valve bypass;

FIG. 2 is a schematic view showing a novel T-stent for use in effecting the distal anastomosis in an aortic valve bypass;

FIG. 3 is a schematic view showing the T-stent of FIG. 2 being used to form the distal anastomosis in an aortic valve bypass;

FIGS. 3A, 3B and 3C are schematic views showing various arrangements for releasably constraining selected portions of the T-stent of FIG. 2;

FIGS. 4-15 are schematic views showing one method for deploying the T-stent of FIG. 2 in the descending aorta so as to form the distal anastomosis in an aortic valve bypass;

FIG. 16 is a schematic view showing an alternative approach for deploying the T-stent of FIG. 2 in the descending aorta;

FIG. 17 is a schematic view showing another alternative approach for deploying the T-stent of FIG. 2 in the descending aorta.

FIGS. 18-28 are schematic views showing another method for deploying the T-stent of FIG. 2 in the descending aorta so as to form the distal anastomosis in an aortic valve bypass;

FIG. 29 is a schematic view showing another novel T-stent for use in effecting the distal anastomosis in an aortic valve bypass; and

FIGS. 30-32 are schematic views showing a method for deploying the T-stent of FIG. 29 in the descending aorta so as to form the distal anastomosis in an aortic valve bypass.

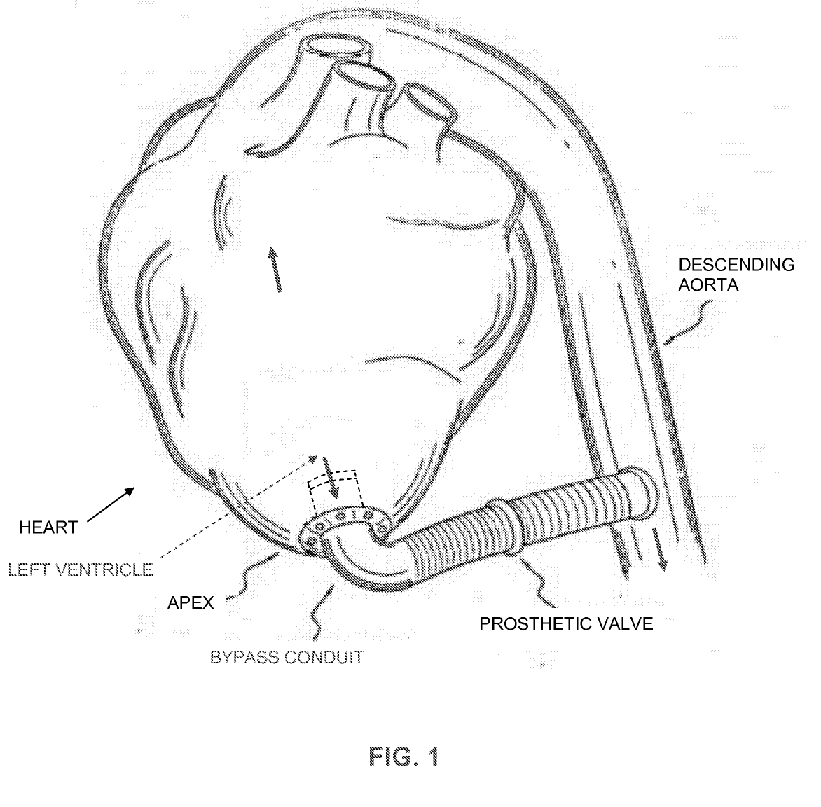

### DETAILED DESCRIPTION OF THE PREFERRED EMBODIMENTS

The present invention comprises a novel method and apparatus for effecting the distal anastomosis in an aortic valve bypass procedure. More particularly, the present invention comprises the provision and use of a novel T-stent to quickly and safely effect the distal anastomosis in an aortic valve bypass procedure, while requiring significantly less access to the anastomosis site and without requiring suturing to the descending aorta. Significantly, hemostasis is effectively maintained at substantially all times, so that the distal anastomosis can be carried out while the heart is beating.

### The Novel T-Stent

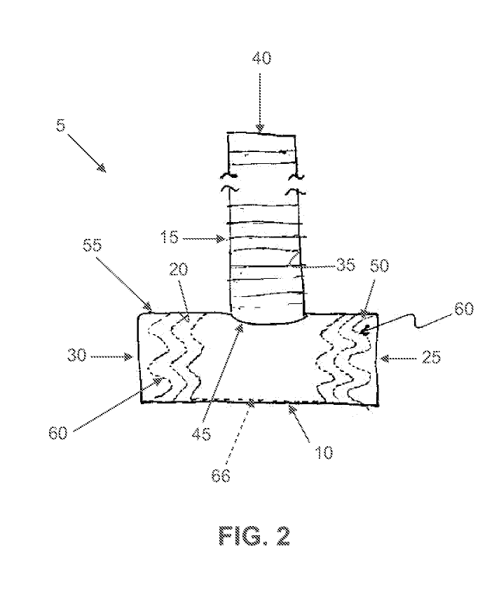

Looking now at FIG. 2, there is shown a novel T-stent 5 which comprises one preferred form of the present invention. T-stent 5 generally comprises a fluid-constraining tube 10 having a fluid-constraining neck 15 extending therefrom. More particularly, tube 10 comprises a lumen 20 having a first opening 25 and a second opening 30. Neck 15 comprises a lumen 35 having a first opening 40 and a second opening 45. Neck 15 is joined to tube 10 so that neck 15 is in fluid communication with tube 10. More particularly, neck 15 is joined to tube 10 intermediate the tube's length so as to bifurcate tube 10 into a first arm 50 and a second arm 55, with second opening 45 of neck 15 communicating with lumen 20 of tube 10. As a result of this construction, fluid entering first opening 25 of tube 10 can exit second opening 30 of tube 10, and fluid entering first opening 40 of neck 15 can exit second opening 30 and first opening 25 of tube 10.

As noted above, T-stent 5 is constructed out of fluid-constraining materials. In one preferred form of the present invention, T-stent 5 is constructed out of woven polyester graft material, e.g. Vascutek GelWeave™. And in one particularly preferred form of the present invention, tube 10 of T-stent 5 is made out of uncrimped (i.e., straight) GelWeave™, and neck 15 (which is joined to tube 10) is made out of crimped GelWeave™. However, it should be appreciated that other materials, including Gore-Tex® fabric or Vascutek Triplex™, can also be utilized.

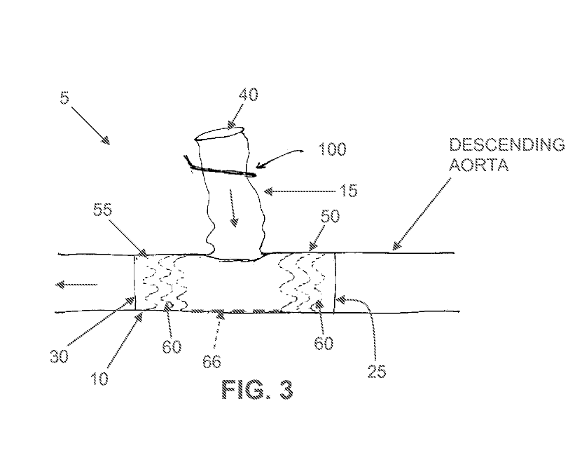

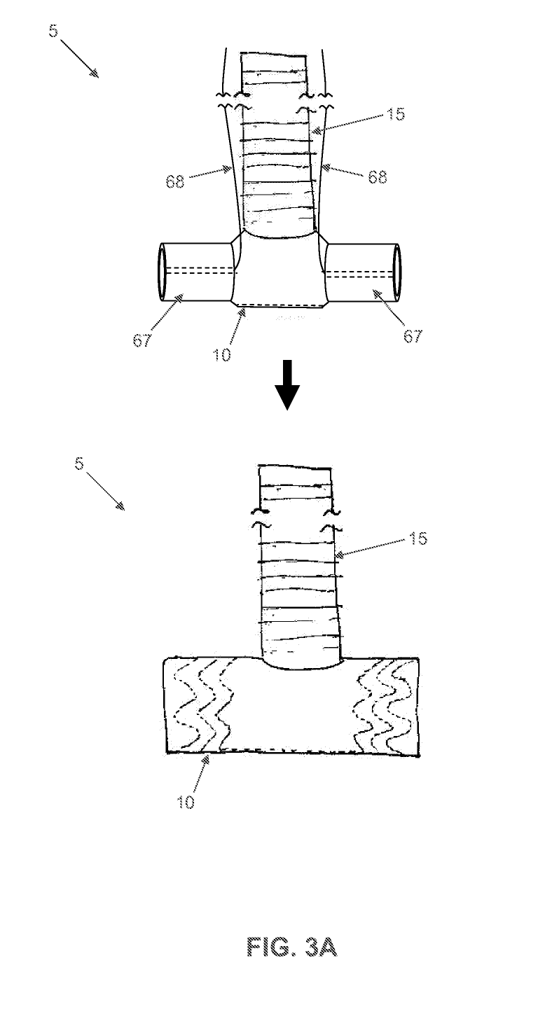

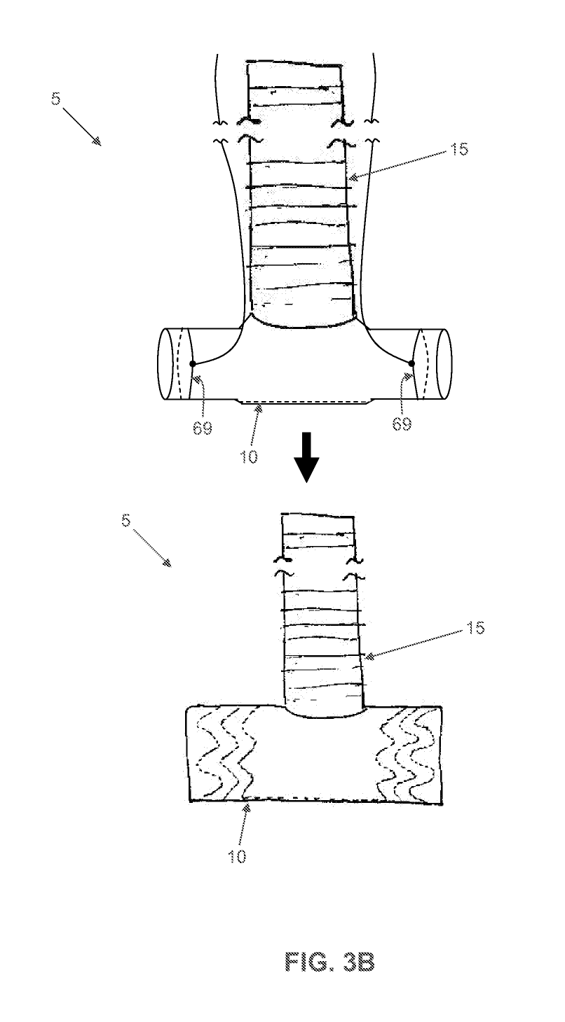

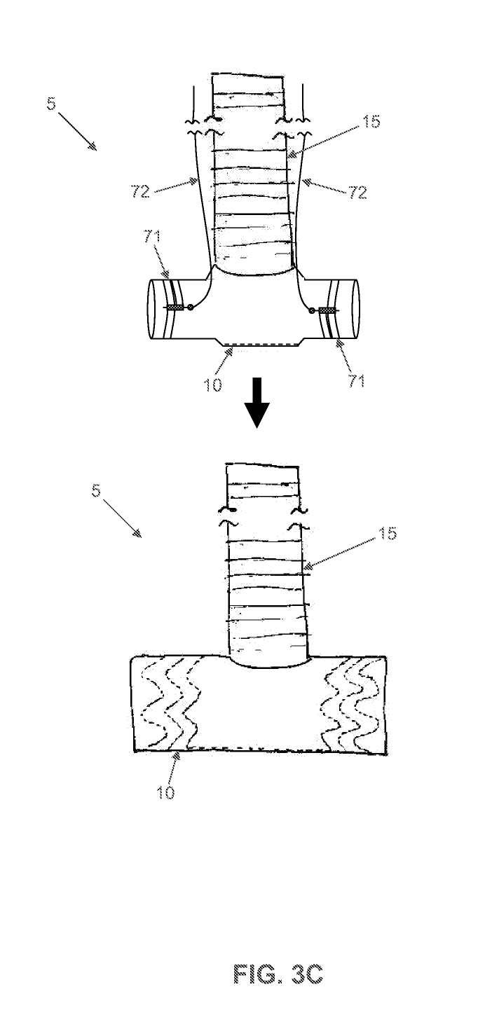

As will hereinafter be discussed in further detail, tube 10 of T-stent 5 is intended to be disposed within the lumen of the descending aorta, with neck 15 extending out through an opening formed in the side wall of the descending aorta, in the manner shown in FIG. 3. In addition, and as will hereinafter also be discussed in further detail, first opening 40 of neck 15 is intended to be connected to the distal end of the bypass conduit. As a result, when the proximal end of the bypass conduit is connected to the left ventricle, blood can be pumped from the left ventricle of the heart into the bypass conduit, into first opening 40 of neck 15 and out second opening 30 of tube 10, so as to create the desired blood flow between the left ventricle of the heart and the descending aorta. In this way, T-stent 5 can serve to effect the distal anastomosis for the aortic valve bypass.

In order to facilitate proper disposition of tube 10 in the descending aorta, at least the portions of tube 10 adjacent to first opening 25 and second opening 30 are biased radially outwardly so that they normally assume a radially-expanded configuration (FIGS. 2 and 3). However, and as will hereinafter be discussed in further detail, the outwardly-biased portions of tube 10 may also be restrained in a radially-contracted condition, in order to facilitate insertion of arms 50 and 55 into the interior of the descending aorta.

More particularly, the portions of tube 10 adjacent to first opening 25 and second opening 30 are preferably outwardly biased by incorporating an array of superelastic alloy (e.g., Nitinol®) or stainless steel Z-stents 60 into the side walls of arms 50 and 55. These spring arrays 60 are of the sort well known in the industry (see, for example, the Cook Gianturco Z-stent). In one preferred embodiment of the present invention, a single length of 0.015″ diameter Nitinol® wire is used to create the spring arrays 60 which outwardly bias each arm of the T-stent. This wire has been heat treated on a mandrel to form a spiral, nested spring on each arm of the T-stent, connected by a substantially straight section 66 extending across the connecting center section of the T-stent. This arrangement avoids any joints in the wire. The Nitinol® or stainless steel Z-stents 60 are preferably attached to the woven polyester graft material of T-stent 5 by adhesive, or by suturing, or by sandwiching the Z-stents 60 between opposing layers of the woven polyester graft material.

As noted above, the outwardly-biased portions of tube 10 may be selectively restrained in a radially-contracted condition, in order to facilitate insertion of arms 50 and 55 into the interior of the descending aorta. This radial restraint is preferably accomplished by restraining the outwardly-biased portions of tube 10 within a tear-away sheath 67 released by a control line 68 (FIG. 3A), or by restraining the outwardly-biased portions of tube 10 with a rip cord 69 (FIG. 3B), or by restraining the outwardly-biased portions of tube 10 with external mechanical clamps 71 released by a control line 72 (FIG. 3C), etc.

As a result of this construction, the outwardly-biased portions of tube 10 may be restrained in a radially-contracted condition during insertion of tube 10 into the interior of the descending aorta, whereupon the restraint(s) may be removed and the outwardly-biased portions of tube 10 thereby permitted to return outboard so as to seat themselves against the interior wall of the descending aorta. In this respect it should be appreciated that T-stent 5 is preferably constructed so that the fully expanded outer diameter (OD) of the two arms 50, 55 is approximately 10-50% greater than the interior diameter (ID) of the descending aorta, so that the two arms 50, 55 will form a close binding fit against the interior wall of the descending aorta.

### Preferred Method For Deploying the T-Stent in the Descending Aorta so as to Create a Distal Anastomosis for an Aortic Valve Bypass

T-stent 5 is preferably deployed in the descending aorta in the following manner so as to create a distal anastomosis for an aortic valve bypass.

1. Arms 50 and 55 of T-stent 5 are radially constrained so as to assume a smaller diameter. As noted above, this may be accomplished by compressing arms 50 and 55 with a tear-away sheath 67 (FIG. 3A), a rip cord 69 (FIG. 3B), external mechanical clamps 71 (FIG. 3C), etc.

2. Access to the descending aorta is created through a small thoracotomy, a thoracoscopy, or other minimally invasive opening in the thoracic cavity.

3. A balloon catheter (Cook Coda® G36042, for example) is fed through neck 15 and first arm 50 of T-stent 5.

4. The physician chooses an acceptable site on the descending aorta.

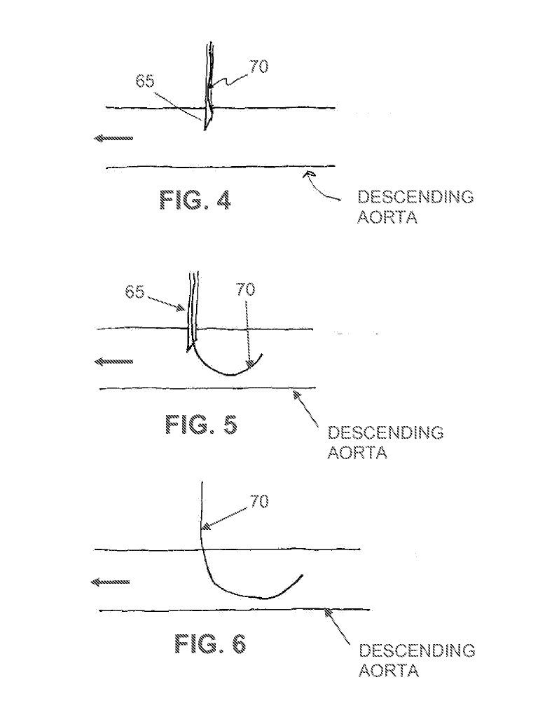

5. A hollow needle 65, containing a relatively stiff, curved guidewire 70, is inserted substantially perpendicularly into the descending aorta at the chosen site. See FIG. 4.

6. The curved guidewire 70 is oriented proximally to the heart and then advanced out of hollow needle 65 so that the guidewire extends toward the heart. See FIG. 5.

7. Hollow needle 65 is withdrawn, leaving curved guidewire 70 in place. See FIG. 6.

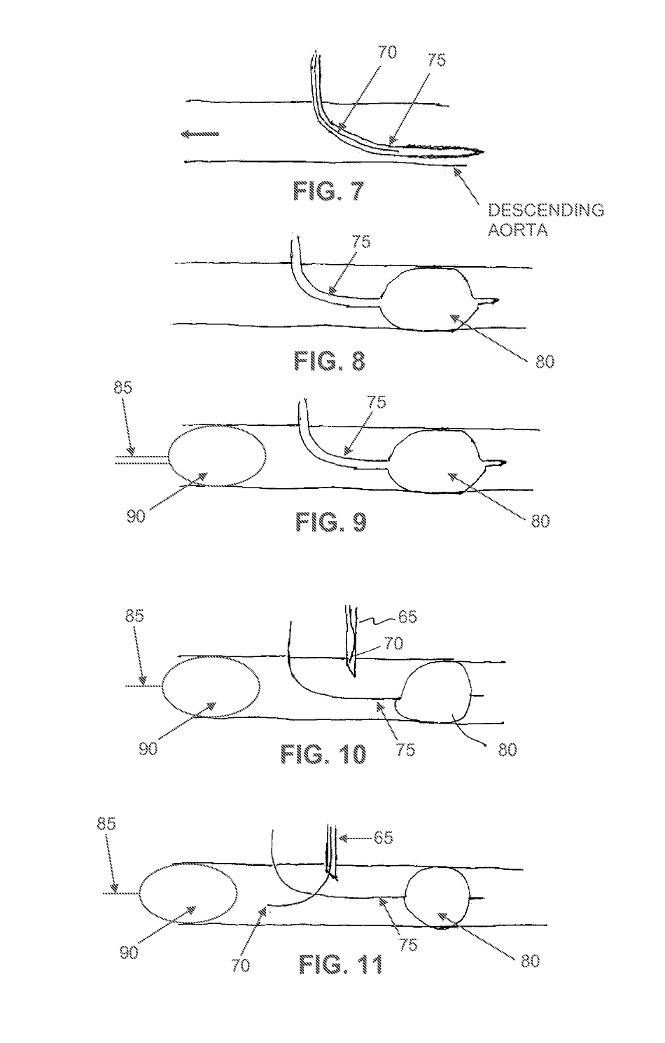

8. The balloon catheter 75, previously fed through neck 15 and first arm 50 of the T-stent (see step 3 above), is advanced over the guidewire. See FIG. 7.

9. The balloon 80 of balloon catheter 75 is inflated so as to occlude the descending aorta. See FIG. 8.

10. A second balloon catheter 85 is introduced into the descending aorta, and its balloon 90 is inflated in the descending aorta, at a location “downstream” from the first balloon 80 so as to occlude the descending aorta at second location. See FIG. 9. As a result, blood flow through the descending aorta is effectively blocked by the two inflated balloons at two locations, i.e., one “upstream” from the entry point of balloon catheter 75 and one “downstream” from the entry point of balloon catheter 75. In one form of the invention, the second balloon catheter 85 is advanced to the anastomosis site by introducing the second balloon catheter into the femoral artery, and then advancing it up the femoral artery, up the iliac branch, and then up the descending aorta.

11. Step 5 is repeated at a second puncture site on the descending aorta, preferably approximately 2 cm proximal (i.e., “upstream”) to the first puncture site, and in any case intermediate inflated first balloon 80 and inflated second balloon 90. See FIG. 10.

12. The curved guidewire 70 introduced at the second puncture site is oriented distally to the heart and then advanced out of the hollow needle 65 so that the guidewire extends away from the heart. See FIG. 11.

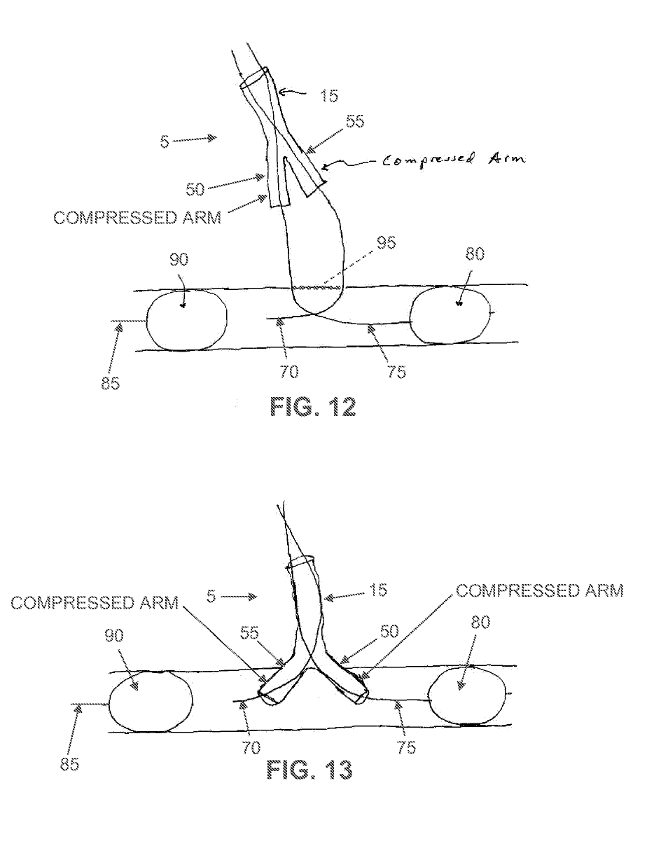

13. Guidewire 70 is passed through second arm 55 and neck 15 of the T-stent, and then the physician cuts a slit 95 between the two puncture sites, thereby creating a slit about 2 cm long. See FIG. 12.

14. Then the two arms of the T-stent are advanced along first balloon catheter 75 and guidewire 70 so that the two arms of the T-stent pass through the aortic slit 95 and into the interior of the descending aorta. For the orientation of balloon catheter 75 and guidewire 70 shown in FIG. 12, the T-stent must be rotated 180° during insertion. See FIG. 13.

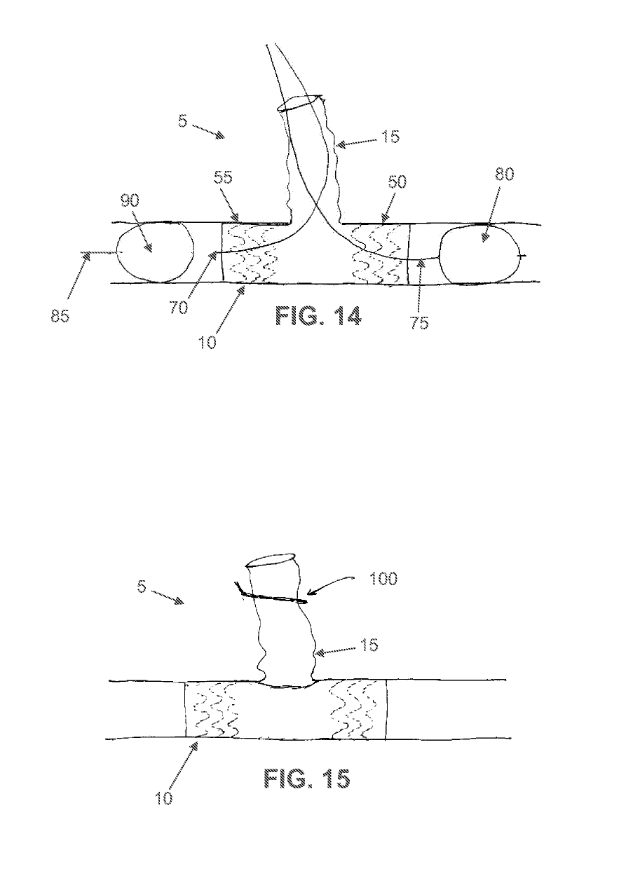

15. Once the two arms 50, 55 of T-stent 5 are disposed somewhat collinear within the descending aorta, and the neck 15 is roughly centered in the slit 95 extending through the side wall of the descending aorta, the two arms 50, 55 are released from their radially-contracted state (e.g., by removing their constraining tear-away sheath 67, or rip cord 69, or external mechanical clamps 71, etc.) and allowed to expand against the inner diameter of the descending aorta. The radial force generated by the Z-stents 60 is sufficient to seal the outer diameter (OD) of the two arms 50, 55 to the inner diameter (ID) of the descending aorta. See FIG. 14.

16. Next, neck 15 of T-stent 5 is blocked off, e.g., with a cross-clamp 100. After the neck of the T-stent has been blocked off, the second balloon 90 is deflated and withdrawn. Then the first balloon 80 is deflated and withdrawn, leaving the T-stent deployed within the aorta. See FIG. 15.

17. At this point, the distal anastomosis is complete. Neck 15 of the T-stent may thereafter be connected to the distal end of the bypass conduit, and cross-clamp 100 removed, as to complete the aortic valve bypass. As a result, as the heart beats, blood is forced out the left ventricle, through the bypass conduit, into first opening 40 of neck 15, and out second opening 30 of tube 10, whereby to deliver oxygenated blood into the descending aorta.

Insertion of arms 50 and 55 of T-stent 5 through the aortic slit 95 and into the lumen of the descending aorta can be aided by a number of instruments such as forceps and endoscopic graspers.

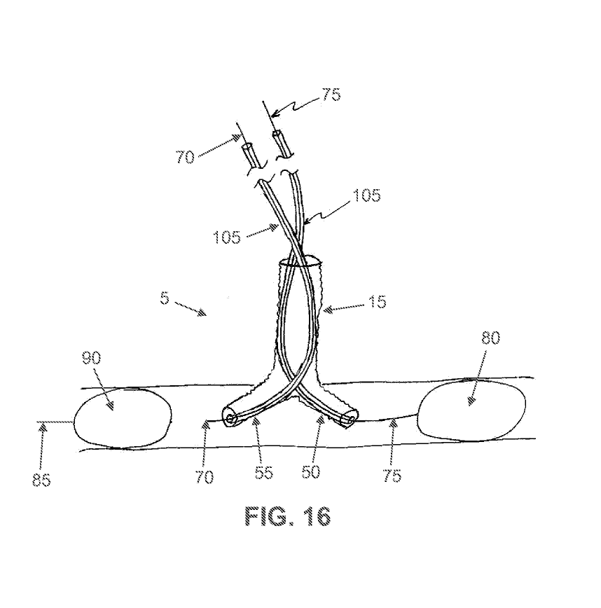

Insertion of the arms of the T-stent into the descending aorta may also be aided by using a positioning sheath 105 (see FIG. 16) placed over balloon catheter 75 and/or guidewire 70. The positioning sheath 105 has proximal and distal ends, where the proximal end is manually controlled to position the distal end of the positioning sheath. The positioning sheath may be movable in the axial and rotational directions with respect to the axis of balloon catheter 75 and/or guidewire 70. The T-stent may be detachably connected to the distal end of the positioning sheath. As such, the proximal end of a positioning sheath 105 can be used to position the T-stent, and particularly an arm of the T-stent, to a desired location within the lumen of the descending aorta. A positioning sheath 105 may be used with one or both of balloon catheter 75 and guidewire 70. For example, positioning sheaths 105 could be used with each of the balloon catheter 75 and guidewire 70, thereby allowing precise positioning of each arm of the T-stent. When the T-stent is positioned in the desired location, the positioning sheaths 105 are detached from the T-stent, such as by detaching a connection. The detachable connection could be a suture, a wire, or other attachment means.

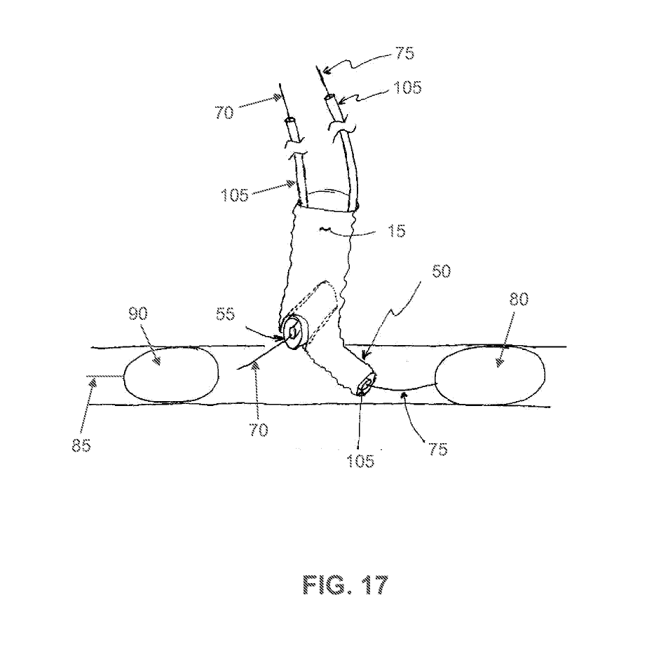

In an alternative approach (see FIG. 17), one arm of the T-stent, such as the second arm 55, could be withdrawn inside the neck of the T-stent until the time for deployment of that second arm into the lumen of the descending aorta. During use, after the first arm 50 of the T-stent has been inserted into the lumen of the descending aorta, the second arm 55 is deployed by pushing the second arm from out of the neck of the T-stent and into the lumen of the descending aorta. In this respect it will be appreciated that second arm 55 is diametrically constrained at this point in the procedure (e.g., by tear-away sheath 67, ripcord 69, mechanical clamps 71, etc.) so that second arm 55 can fit easily within neck 15 of the T-stent. This alternative configuration could use a positioning sheath 105, as described above, to push the inverted second arm 55 from inside the neck of the T-stent into the lumen of the descending aorta. This alternative configuration can ease insertion of the arms 50, 55 of the T-stent through the slit 95 in the aorta wall.

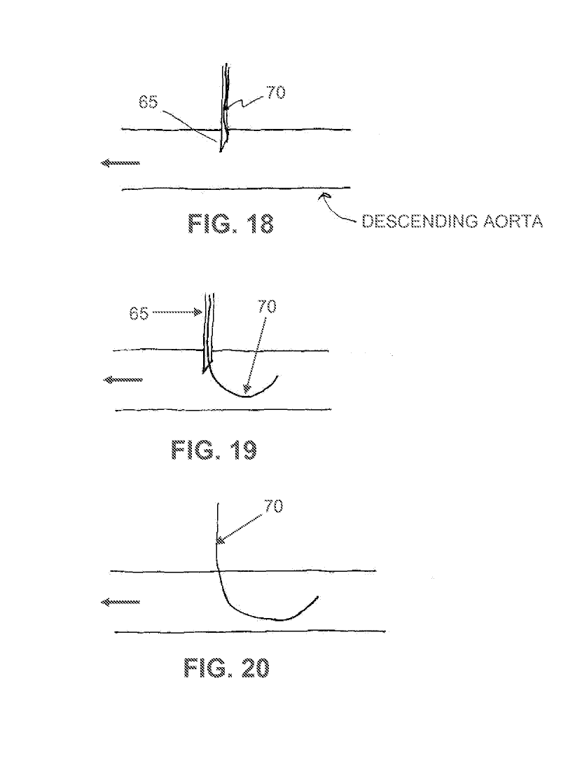

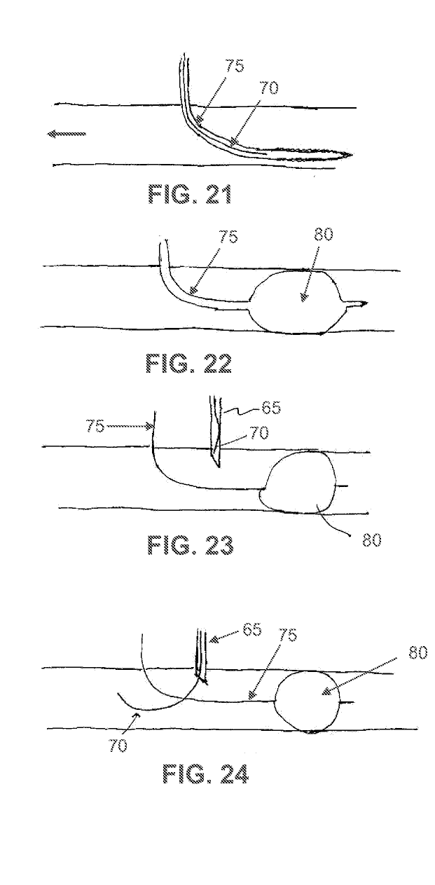

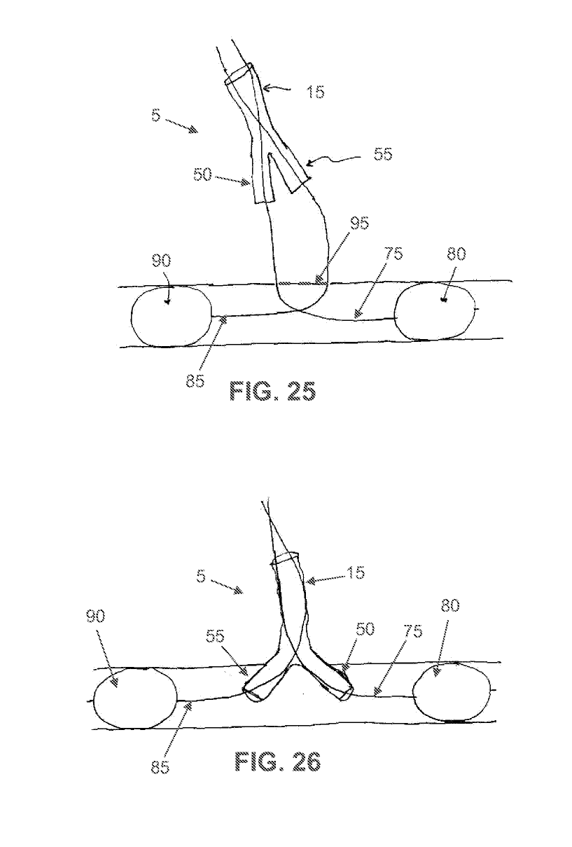

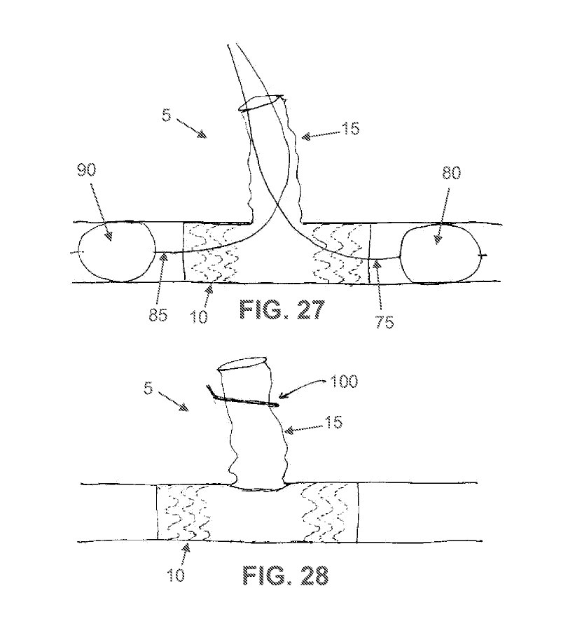

FIGS. 18-28 illustrate an alternative form of the invention wherein second balloon catheter 85 is introduced into the distal anastomosis site via a side wall puncture in the descending aorta, rather than via the aformentioned femoral artery approach. More particularly, with this form of the invention, hollow needle 65 and guidewire 70 are introduced into the descending aorta at a first puncture site (FIG. 18); guidewire 70 is oriented proximally to the heart and advanced out of needle 65 (FIG. 19); needle 65 is withdrawn (FIG. 20); a first (“upstream”) balloon catheter 75 is advanced into the descending aorta using guidewire 70 (FIG. 21); first balloon 80 is inflated (FIG. 22) so as to occlude the descending aorta; a hollow needle 65 and guidewire 70 are introduced into the descending aorta at a second puncture site (FIG. 23); guidewire 70 is oriented distally to the heart and advanced out of the needle (FIG. 24); a second (“downstream”) balloon catheter 85 is advanced into the descending aorta using guidewire 70 and its balloon 90 is inflated (FIG. 25) so as to occlude the descending aorta at a second location, and the slit 95 is formed in the side wall of the descending aorta; the T-stent 5 (with arms 50 and 55 radially contracted) is advanced into position within the descending aorta (FIG. 26); arms 50 and 55 of the T-stent are allowed to assume their radially-expanded condition (FIG. 27); neck 15 of the T-stent is clamped, and the balloons 80, 90 are deflated (FIG. 28); and finally neck 15 is connected to the bypass conduit, whereby to complete the aortic valve bypass.

### Alternative T-Stent

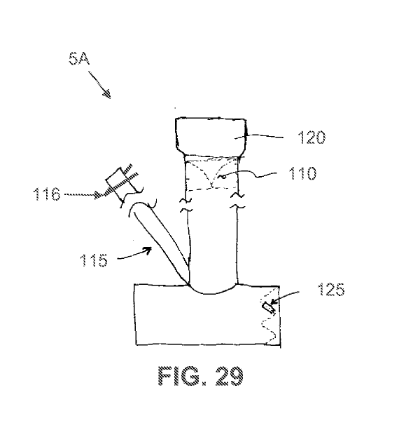

Looking next at FIG. 29, there is shown a T-stent 5A which comprises an alternative form of the present invention. T-stent 5A is substantially the same as the T-stent 5 previously discussed, except as will hereinafter be discussed.

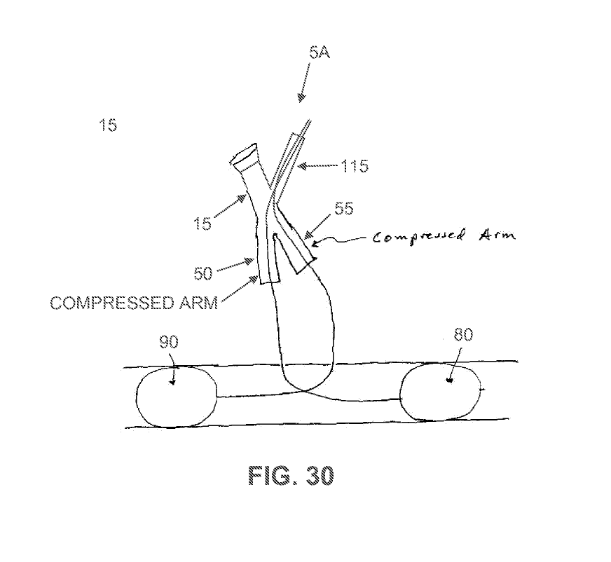

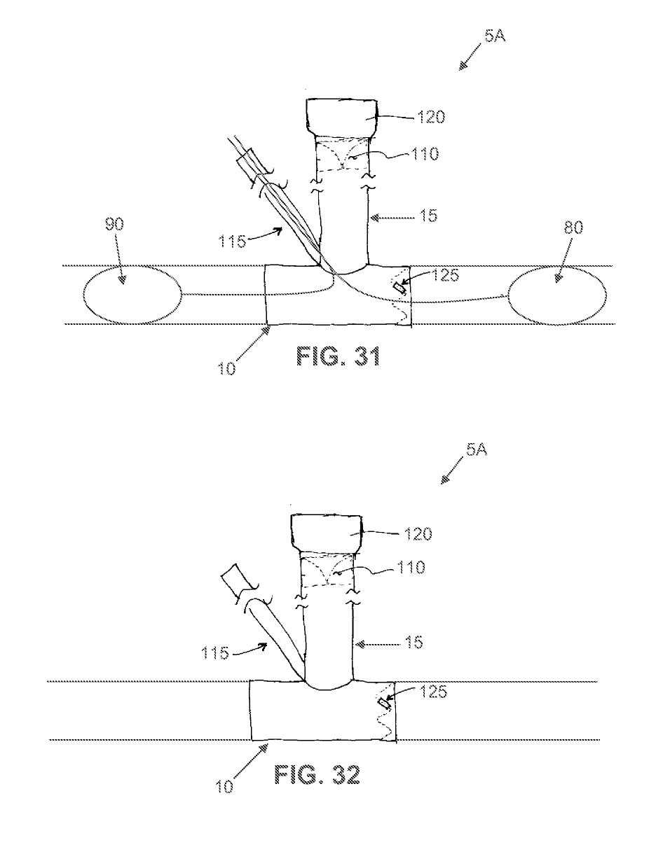

More particularly, in this form of the invention, T-stent 5A also includes a side branch 115 which provides access to the second opening 45 of neck 15 without passing through first opening 25 of neck 15 (FIG. 2). With a side branch 115 present on the neck 15 of the T-stent, opening 25 of neck 15 can be blocked off, e.g., with a clamp, or with a valve 110 (as discussed below), while the balloon catheters (and/or guidewire) can be fed through the side branch 115 of the T-stent, and then into their respective arms of the T-stent. Installation then proceeds as outlined above (see FIGS. 30-32). Once the T-stent is fully deployed in the body, and access through side branch 115 is no longer necessary, side branch 115 is permanently sealed using suture, a clip, staples, etc.

Side branch 115 is preferably sized so as to be much smaller in diameter than neck 15, i.e., just large enough to accommodate guidewire 70, balloon catheter 75, etc., and much smaller than the relatively large blood passageway needed in neck 15 to accommodate the substantial blood flow required for a successful aortic valve bypass. As a result, the provision and use of a relatively small diameter side branch 115 allows blood loss through the T-stent to be minimized during the time when balloons 80 and 90 are deflated and withdrawn and before the T-stent can be clamped off.

In one preferred form of the invention, side branch 115 is pre-clamped with a removable clamp 116.

And, if desired, side branch 115 can include additional sealing means to seal around guidewire 70, balloon catheter 75, etc. Significantly, the design constraints on such sealing means are significantly eased since the sealing means need not be removed from the T-stent in order for the T-stent to be become utilized for bypass flow, since the bypass flow is through neck 15 and not through side branch 115.

T-stent 5A may also include a pre-installed removable clamp or, alternatively, a prosthetic valve 110 (FIGS. 29-32) pre-installed in neck 15, whereby to eliminate the need for cross-clamping neck 15 as previously disclosed.

And T-stent 5A may include a connector 120 for attaching neck 15 to the bypass conduit. This connector 120 may be a male-female slip connector of the sort taught in FIG. 15 of U.S. Pat. No. 7,510,561, issued Mar. 31, 2009 to Richard M. Beane et al. for APPARATUS AND METHOD FOR CONNECTING A CONDUIT TO A HOLLOW ORGAN (Attorney's Docket No. CORREX-033058-000005), which patent is hereby incorporated herein by reference; or a snap-together coupling with self-sealing capability on at least one side of the coupling. If desired, radiopaque markers 125 may also be provided. Such radiopaque markers can be extremely useful for locating the T-stent when fluoroscopy is available.

Where a prosthetic valve 110 is pre-installed within the neck of the T-stent, it is preferred that side branch 115 also be provided so that the side branch 115 allows the prosthetic valve to remain undisturbed throughout installation of the T-stent.

### Use of the Present Invention for Other Applications

As disclosed above, the present invention may be used for effecting a distal anastomosis for an aortic valve bypass. However, it should be appreciated that the present invention can also be used for a distal anastomosis for any bypass procedure, or for substantially any joinder of one vessel to another vessel.

### Further Modifications

It will be understood that many additional changes in the details, materials, steps and arrangements of parts, which have been herein described and illustrated in order to explain the nature of the invention, may be made by those skilled in the art while remaining within the principles and scope of the present invention.

What is claimed is:

1. A connector for joining a first hollow structure to the side wall of a second hollow structure, the connector comprising:
a fluid-constraining tube having a fluid-constraining neck extending therefrom, wherein the tube comprises a lumen having a first opening and a second opening and the neck comprises a lumen having a first opening and a second opening, the neck being joined to the tube so that the neck is in fluid communication with the tube intermediate the length of the tube, such that fluid entering the first opening of the tube can exit the second opening of the tube, and fluid entering the first opening of the neck can exit the second opening of the tube;
at least the portions of the tube adjacent to the first opening of the tube and the second opening of the tube being biased radially outwardly so that they normally assume a radially-expanded configuration, but being capable of being restrained in a radially-contracted configuration, wherein the tube is sized so that, when it is in its radially-expanded configuration, it has an outer diameter which is larger than the inner diameter of the second hollow structure.

2. A connector according to claim 1 wherein a spring is disposed adjacent to at least the portions of the tube adjacent to the first opening of the tube and the second opening of the tube so as to bias at least those portions of the tube radially outwardly so that they normally assume a radially-expanded configuration.

3. A connector according to claim 2 wherein the spring comprises a first cylindrical portion adjacent to the first opening of the tube and a second cylindrical portion adjacent to the second opening of the tube.

4. A connector according to claim 3 wherein the spring comprises a substantially straight portion connecting the first cylindrical portion to the second cylindrical portion.

5. A connector according to claim 2 wherein at least one of the first cylindrical portion and the second cylindrical portion comprises a Z-stent.

6. A connector according to claim 5 wherein the Z-stent comprises a material selected from the group consisting of a superelastic alloy and stainless steel.

7. A connector according to claim 1 wherein the connector comprises woven polyester graft material.

8. A connector according to claim 7 wherein the tube comprises uncrimped woven polyester graft material and the neck comprises crimped woven polyester graft material.

9. A connector according to claim 1 further comprising a restraint for holding the tube in its radially-contracted configuration.

10. A connector according to claim 9 wherein the restraint comprises at least one from the group consisting of a tear-away sheath, a rip cord and external mechanical clamps.

11. A connector according to claim 1 wherein a side branch provides access to the interior of the neck.

12. A connector according to claim 11 wherein the side branch has a diameter significantly smaller than the diameter of the neck.

13. A connector according to claim 11 further comprising a removable clamp pre-installed on the side branch.

14. A connector according to claim 1 wherein a prosthetic valve is disposed in the neck.

15. A connector according to claim 14 wherein the prosthetic valve is arranged to permit fluid to flow from the first opening of the neck to the second opening of the neck but to prevent fluid from flowing in the reverse direction.

16. A connector according to claim 1 wherein a removable clamp is pre-installed on the neck.

17. A connector according to claim 1 wherein the neck comprises a connector for connecting the neck to the first hollow structure.

18. A connector according to claim 1 wherein the first hollow structure comprises a bypass conduit and the second hollow structure comprises the descending aorta.

19. A connector according to claim 1 wherein the first hollow structure comprises the left ventricle of the heart, the neck comprises a bypass conduit, and the second hollow structure comprises the descending aorta.

20. A method for joining a first hollow structure to the side wall of a second hollow structure, the method comprising:
providing a connector comprising:
a fluid-constraining tube having a fluid-constraining neck extending therefrom, wherein the tube comprises a lumen having a first opening and a second opening and the neck comprises a lumen having a first opening and a second opening, the neck being joined to the tube so that the neck is in fluid communication with the tube intermediate the length of the tube, such that fluid entering the first opening of the tube can exit the second opening of the tube, and fluid entering the first opening of the neck can exit the second opening of the tube;
at least the portions of the tube adjacent to the first opening of the tube and second opening of the tube being biased radially outwardly so that they normally assume a radially-expanded configuration, but being capable of being restrained in a radially-contracted configuration, wherein the tube is sized so that, when it is in its radially-expanded configuration, it has an outer diameter which is larger than the inner diameter of the second hollow structure;
restraining the tube in its radially-contracted configuration;
selecting a location on the side wall of the second hollow structure;
forming an opening in the side wall of the second hollow structure at the selected location;
positioning the connector so that the tube resides within the interior of the second hollow structure and the neck extends out of the side wall of the second hollow structure;
allowing the tube to expand back into its radially-expanded configuration; and
connecting the neck to the first hollow structure.

21. A method according to claim 20 wherein fluid flow through the second hollow structure is blocked upstream of the selected location prior to positioning the tube in the interior of the second hollow structure.

22. A method according to claim 21 wherein a balloon is erected in the second hollow structure to block fluid flow.

23. A method according to claim 22 wherein the balloon is inserted into the interior of the second hollow structure via the opening.

24. A method according to claim 20 wherein fluid flow through the second hollow structure is blocked downstream of the selected location prior to positioning the tube in the interior of the second hollow structure.

25. A method according to claim 24 wherein a balloon is erected in the second hollow structure to block fluid flow.

26. A method according to claim 24 wherein the balloon is inserted into the interior of the second hollow structure via a second opening.

27. A method according to claim 20 wherein the first hollow structure comprises a bypass conduit and the second hollow structure comprises the descending aorta.

28. A method according to claim 20 wherein the first hollow structure comprises the left ventricle of the heart, the neck comprises a bypass conduit, and the second hollow structure comprises the descending aorta.
